(19) United States
(12) Patent Application Publication
(10) Pub. No.: US 2018/0078164 A1
(43) Pub. Date: Mar. 22, 2018

(54) BRAIN ACTIVITY DETECTION SYSTEM, DEVICES AND METHODS UTILIZING THE SAME

(71) Applicant: Intel Corporation, Santa Clara, CA (US)

(72) Inventors: VIVEK K. MENON, Overland Park, KS (US); INDIRA NEGI, San Jose, CA (US); THAO-VY NGUYEN, San Francisco, CA (US)

(73) Assignee: Intel Corporation, Santa Clara, CA (US)

(21) Appl. No.: 15/269,615

(22) Filed: Sep. 19, 2016

(51) Int. Cl. A61B 5/04; A61B 5/00; A61B 5/048; A61B 5/0478

(52) U.S. Cl. CPC A61B 5/04017; A61B 5/6898; A61B 5/6803; A61B 5/0478; A61B 5/7257; A61B 5/048; A61B 5/4809

(57) ABSTRACT

Brain activity detection technologies are disclosed. In some embodiments the technologies include a brain activity detection system that includes a sensor block including that includes at least one reference electrode and at least one sense electrode. The reference and sense electrodes are configured to produce analog reference and analog sense signals that are indicative of activity of a brain of a user. A signal processing block may also be included, and is configured to generate at least one digital differential signal based at least in part on the analog reference and analog sense signals. At least one processor may convert the digital differential signal(s) in said time domain to one or more frequency domain digital differential signals. Signal processing on the frequency domain digital differential signal may be performed to determine health information of a user. Devices, methods, and computer readable media utilizing such technologies are also disclosed.

### FIELD

The present disclosure generally relates brain activity detection technologies, and in particular, to brain activity detection systems. Devices and methods utilizing such technologies are also described.

### BACKGROUND

In recent years interest has grown in the development of consumer fitness devices that enable consumers to monitor various aspects of their health, such as their heart rate, blood pressure, blood oxygen content, their overall activity level (e.g., motion), and the like. As consumer awareness of the health related impacts of stress, lack of sleep, and/or poor quality sleep on the human brain has grown (particularly as people have grown increasingly busy), interest has grown in particular in the development of devices that enable consumers to monitor and track factors concerning the health of their brain. In particular, interest has grown in consumer devices that enable users to track their sleep patterns outside of a clinical environment (e.g., in their home). Various consumer sleep monitoring devices have therefore been introduced into the marketplace in an effort to enable consumers to monitor and evaluate the quality of their sleep.

Some existing sleep monitoring devices rely on motion, pressure, and/or heart rate data (e.g., obtained from motion, pressure, and/or heart rate sensors) to determine whether a wearer of the device is asleep and, if so, to determine which sleep stage the wearer is in. For example, some wearable fitness tracking devices utilize accelerometer data and/or heart rate data to determine whether or not the wearer is sleeping, e.g., based on a relatively lack of wrist movement and/or a reduction in heart rate. Such devices may also use the same type of data to determine what sleep stage the wearer is in, how long they are in a particular sleep stage, or the like.

While existing consumer sleep monitoring devices are useful to some extent, it has been shown that certain sleep stages are not highly correlated to wrist movement, heart rate, and/or pressure data. For example the REM stage of sleep is not highly correlated with motion or heart rate. Devices relying on such data may therefore inaccurately determine which sleep state a person is in, and for how long. Moreover, existing consumer fitness tracking devices may not directly measure electrophysiological signals from the brain, and therefore may provide inaccurate information concerning the health of the brain of a user.

### BRIEF DESCRIPTION OF THE DRAWINGS

Features and advantages of embodiments of the claimed subject matter will become apparent as the following Detailed Description proceeds, and upon reference to the Drawings, wherein like numerals depict like parts, and in which:

FIG. 1 is a block diagram of one example of a brain activity detection system consistent with the present disclosure.

FIG. 2 is a block diagram of another example of a brain activity detection system consistent with the present disclosure.

FIG. 3 is a block diagram of one example of a brain activity detection device consistent with the present disclosure.

FIG. 4 is a block diagram of one example of a brain activity detection device consistent with the present disclosure.

FIG. 5 provides an overview of electroencephalogram (EEG) electrode nomenclature under the “10-20 system.”

FIG. 6 is a flow chart of example operations of a method of detecting brain activity consistent with the present disclosure.

FIG. 7 is an illustration of a correlation between detected EEG signals and various human sleep stages.

FIGS. 8A-8D illustrate one example of a sleep accessory consistent with the present disclosure.

### DETAILED DESCRIPTION

Embodiments of the present disclosure include technologies (e.g., systems, devices, and methods) for detecting activity of the brain of an animal, such as the human brain. In some embodiments the brain activity detection system includes a sensor block including at least one differential sensor pair. The at least one differential sensor pair includes at least one reference electrode and at least one sense electrode that are configured to produce analog reference and analog sense signals respectively, wherein the analog reference and analog sense signals indicative of activity of a brain of a user. The brain activity detection system may further include a signal processing block coupled to the sensor block. The signal processing block includes circuitry to generate at least one digital differential signal based at least in part on the analog reference and analog sense signals, wherein the at least one digital differential signal is in a time domain. The system may further include at least one processor in communication with the signal processing block. The at least one processor may be configured to convert the at least one digital differential signal in the time domain to a frequency domain digital differential signal that is indicative of health information corresponding to the brain. Signal analysis may be performed on the frequency domain digital differential signal to determine health information related to the brain of the user. The health information may be, for example, an indication of brain activity, stress level, a sleep/awareness state, or the like. In some embodiments the at least one reference electrode and the at least one sense electrode in the system are or include a capacitive electrode.

As used herein, the term “and/or” when used in the context of the two elements (A) and (B), means (A) or (B), or (A) and (B). Likewise, the term “and/or” when used in the context of three or more elements such as (A), (B), and (C), means (A) or (B) or (C), (A) and (B) or (C), (A) and (C) or (B), (B) and (C) or (A), or (A), (B), and (C).

The present disclosure may utilize perspective-based descriptions (e.g., top, bottom, in, out, over, under, and the like) to describe the relative position of one element to another. It should be understood that such descriptions are merely used to for the sake of clarity and ease of understanding, and are not intended to restrict the application of embodiments described herein to any particular orientation unless expressly indicated otherwise.

As used herein the phrases “in an embodiment” and “in embodiments” are used interchangeably to refer to one or more of the same or different embodiments. Furthermore the terms “comprising,” “comprises,” “including,” “includes,” “having” and the like, are interchangeably used herein in connection with descriptions of embodiments of the present disclosure, and are synonymous.

The terms, “first,” “second,” “third,” and the like are used herein to distinguish between similar elements, and not necessarily for describing a particular sequential or chronological order. It should be understood that such terms may be interchangeably used in appropriate circumstances, such that the aspects of the present disclosure may be operable in an order other than which is explicitly described.

As used herein the terms “substantially” and “about” when used in connection with a value or range of values mean plus or minus 5% of the denoted value or the end points of the denoted range.

The terms “coupled” and “connected” are used in connection with the description of various embodiments. Depending on the context, such terms may mean that two or more elements are in direct physical, electrical, or optical contact. Alternatively, “coupled” may also mean that two or more elements are not in direct contact with one another, but still cooperate or interact with one another in some described fashion.

The brain activity detection technologies described herein may be implemented using one or more electronic devices. The terms “device,” “devices,” “electronic device” and “electronic devices” are interchangeably used herein to refer individually or collectively to any of the large number of electronic devices that may be used as or in a brain activity detection system consistent with the present disclosure.

Non-limiting examples of devices that may be used in accordance with the present disclosure include any kind of mobile device and/or stationary device, such as cameras, cell phones, computer terminals, desktop computers, electronic readers, facsimile machines, kiosks, netbook computers, notebook computers, internet devices, payment terminals, personal digital assistants, media players and/or recorders, servers, set-top boxes, smart phones, tablet personal computers, ultra-mobile personal computers, wired telephones, combinations thereof, and the like. Such devices may be portable or stationary. Without limitation, in some embodiments the brain activity detection technologies described herein are implemented in or with one or more mobile electronic devices, such as one or more cellular phones, desktop computers, electronic readers, laptop computers, set-top boxes, smart phones, tablet personal computers, televisions, wearable electronic devices (e.g., belt buckles, clip on devices, headpieces, eyewear, pins, jewelry, and/or ultra-mobile personal computers. In some instances, the brain activity detection technologies herein are implemented in or with a smart phone, a wearable device, a sleep accessory (e.g., a pillow, a sheet, sleepwear, etc.) or a combination thereof.

The term “eyewear” is used herein to generally refer to objects that are worn over one or more eyes of a user (e.g., a human). Non-limiting examples of eyewear include eye glasses (prescription or non-prescription), sun glasses, goggles (protective, night vision, underwater, or the like), a face mask, combinations thereof, and the like. In many instances, eyewear may enhance the vision of a wearer, the appearance of a wearer, or another aspect of a wearer. Similarly, the term “headwear” is generally used to refer to objects that are worn on the head of a user (e.g., a human). Non-limiting examples of headwear include eyewear as discussed above, face masks, helmets, gaming and virtual reality headsets, balaclavas, turbans, head scarves, combinations thereof, and the like.

As used in any embodiment herein, the term “module” may refer to software, firmware, circuitry, and combinations thereof, which is/are configured to perform one or more operations consistent with the present disclosure. Software may be embodied as a software package, code, instructions, instruction sets and/or data recorded on non-transitory computer readable storage mediums, which when executed may cause an electronic device to perform operations consistent with the present disclosure, e.g., as described in the methods provided herein. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, software and/or firmware that stores instructions executed by programmable circuitry. The modules may, collectively or individually, be embodied as circuitry that forms a part of one or more devices, as defined previously. In some embodiments one or more of the modules described herein may be in the form of logic that is implemented at least in part in hardware to perform brain activity detection operations consistent with the present disclosure.

As briefly explained in the background, interest has grown in development of devices that enable consumers to monitor and track factors concerning the health of their brain. In particular, interest has grown in consumer devices that enable users to track their brain activity (e.g., their sleep patterns, quality of their sleep, stress level, etc.) outside of a clinical environment (e.g., in their home). With regard to sleep tracking, various consumer devices have been introduced into the marketplace in an effort to enable consumers to monitor and evaluate the quality of their sleep. Such devices often operate by correlating detected parameters such as user heart rate, breathing rate, wrist/limb motion, auditory signals (e.g., breathing/snoring detection), and the like to a level of user consciousness. For example, some devices utilize accelerometers to measure wrist movement and, based on the amount of detected movement, make a determination as to whether or not the user of the device is sleeping. Although such parameters may be useful in determining when a person is asleep in general, they often produce inaccurate results with regard to determining what sleep stage a person is in, and for how long. For example, the rapid eye movement (REM) stage of sleep has been shown to be not highly correlated with either motion or heart rate and, therefore, devices relying on such parameters to determine when a user is in REM sleep may produce inaccurate results. Moreover, such devices in general do not provide or rely on direct measurements of user brain activity.

In the clinical context, electroencephalography (EEG) is often used to detect problems with a patient's brain, and/or to evaluate the quality and quantity of a patient's sleep. In general, an EEG test involves the use of skin contact electrodes or other sensors to detect electrophysiological signals produced by the brain. The resulting electroencephalogram produced by the test is a measurement of sum signal emanating from changes in voltage of the membranes of nerve cells in the brain. Depending on the nature of the test, the number of electrodes used and their placement on the patient's body (and in particular, their head) may vary. For example in the context of a traditional sleep study analysis (SSA), a clinician may place a large number of gel or other electrodes in direct contact with the scalp of a patient in a controlled environment, such as a sleep study laboratory. The patient is then instructed to sleep, during which the electrodes are used to measure the electrical activity of the patient's brain. The resulting electroencephalogram may then be used by the physician to determine how long the patient slept, the quality of their sleep (e.g., which sleep stages the patient entered and for how long), and various other parameters that may help the physician determine whether the patient is suffering from one or more neurological or sleep related disorders.

While traditional EEG machines such as those used in the context of a sleep study analysis can provide a highly accurate depiction of the electrical activity of the human brain, they generally utilize a large number of gel or other direct contact electrodes, making them very inconvenient to use by individuals other than clinicians, and/or in the context of a consumer device. With that in mind, the inventors have recognized that useful brain activity information may be detected from users using EEG technology, but without the need for a large number of gel or other direct contact electrodes. For example and as will be described below, useful signals that are indicative of user brain activity that in turn can be correlated to useful health related information (e.g., when a user is asleep and which sleep stage they are in) may be obtained using relatively few electrodes, including electrodes that do not have to be in direct contact with the user's scalp.

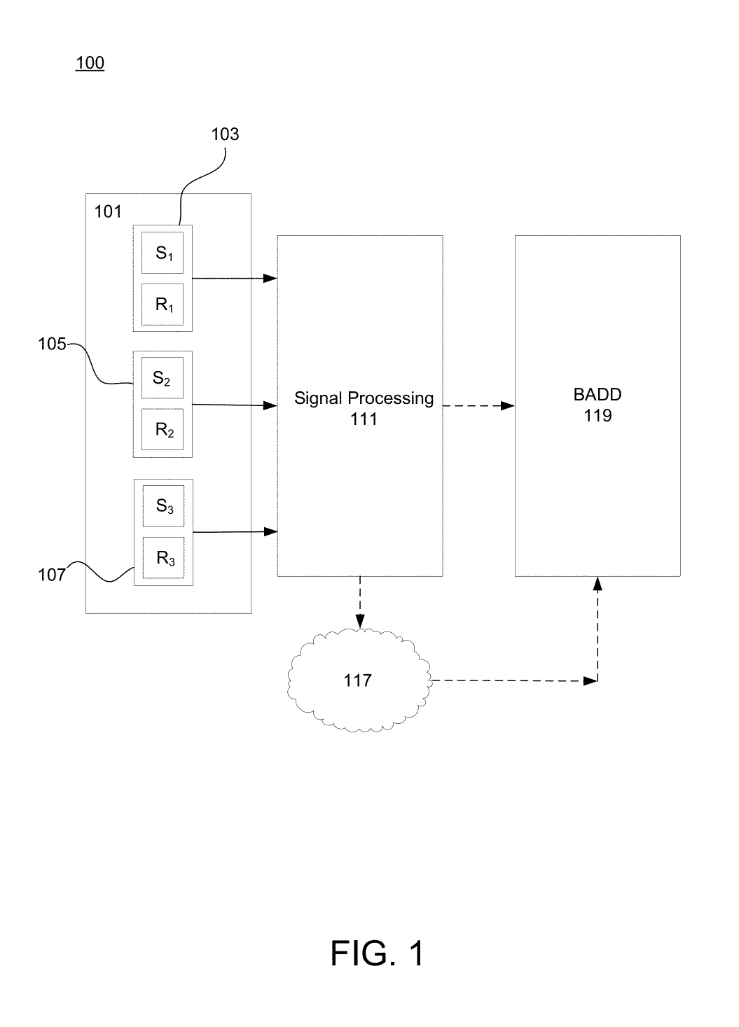

With the foregoing in mind reference is made to FIG. 1 which is a block diagram of one example of a brain activity detection system (BADS) consistent with the present disclosure. As shown, BADS 100 includes sensor block 101, signal processing block 111, and brain activity detection device (BADD) 119. In general, sensor block 101 includes at least one differential sensor pair, wherein each differential sensor pair includes a sense electrode and a reference electrode. That concept is shown in FIG. 1, which depicts sensor block as including a first differential sensor pair 103, a second differential sensor pair 105, and a third differential sensor pair 107. Each differential sensor pair in sensor block 101 includes at least one sense electrode (S), and at least one reference electrode (R). This concept is shown in the embodiment of FIG. 1, which depicts sensor block 100 as including a first differential sensor pair 103 including a first sense electrode (S₁) and a first reference electrode (R₁), a second differential sensor pair 105 including a second sense electrode (S₂) and a second reference electrode (R₂), and a third differential sensor pair 107 including a third sense electrode (S₃) and a third reference electrode (R₃).

In general, the sense and reference electrodes are each configured to produce an electrical signal that is indicative of brain activity (e.g., of a human brain) proximate to their location. That is, when BADS 100 is in use, the sense and reference electrodes within each differential sensor pair may be placed on or in proximity to different locations of the head of a user. Thereafter, the sense and reference electrodes may produce electrical signals indicative of brain activity in regions of the user's brain that are proximate their respective locations.

It is noted that while FIGS. 1-3 depict various embodiments in which differential sensor pairs 103, 105, 107 include a single sense and single reference electrode, such a configuration is not required. Indeed the present disclosure envisions embodiments in which each differential sensor pair includes a plurality of sense and reference electrodes. In such instances the plurality of sense electrodes may be substantially collocated with one another when the BADS is in use. Likewise, the plurality of reference electrodes may be substantially collocated with one another when the BADS is in use. That is, the plurality of sense and reference electrodes may be positioned proximate to one another at a relatively common location along the body of a user, such as at particular locations on a user's head. Use of a plurality of sense and reference electrodes may be beneficial for example, by enabling stronger detection of biological signals produced by the brain.

It is noted that while FIG. 1 depicts BADS 100 as including three differential sensors pairs, a larger or smaller number of differential sensor pairs may also be used depending on the application. For example, in some embodiments only one differential sensor pair may be used. In other embodiments (e.g., where BADS is to be used for to monitor a user's sleep), a plurality (e.g., 2, 3, 4 or more) differential sensor pairs may be used. In any case additional electrodes (e.g., in conjunction with or independent of a differential sensor pair) may also be provided. In such instances, the additional electrodes may be configured to produce an alternative reference or an alternative sense signal which may be substituted for a primary reference or sense signal, e.g., in the event that the primary sense/reference signal is determined to be unsuitable due to noise or other factors.

The nature and type of electrodes used as the sense and reference electrodes is not particularly limited, and any suitable type of electrode may be used for such components so long as it is capable of detecting electrophysiological signals produced by the brain (i.e., EEG signals). Accordingly, the sense and reference electrodes in some embodiments are each in the form of an EEG electrode. As non-limiting examples of suitable EEG electrodes that may be used as sense and reference electrodes consistent with the present disclosure, mention is made of contact electrodes and non-contact electrodes. As used herein, a contact electrode is an electrode that needs to be placed in direct contact with skin (e.g., of the scalp) to detect electrophysiological (EEG) signals produced by the brain. In contrast, a “non-contact” electrode is an electrode that can detect electrophysiological (EEG) signals produced by the brain without the need to be in direct contact with skin. Non-limiting examples of contact electrodes include gel contact electrodes (e.g., electrodes that utilize a conductive gel to facilitate electrical contact with the skin) and dry contact electrodes.

Non-limiting examples of non-contact electrodes that may be used as sense and/or reference electrodes include capacitive electrodes. In some embodiments, non-contact electrodes in the form of capacitive electrodes that detect changes in electrical fields that are the result of, for example, the production of electrical signals within the brain, and produce a corresponding electrical signal. For example, capacitive electrodes can detect microvolt changes resulting from changes in the magnitude of action potential currents running through neurons in the brain. One specific non-limiting example of a suitable non-contact electrode is the capacitive electrode sold by the PLESSEY® corporation under part number PS25255. As may be appreciated, the capacitive electrodes described above may not require the application of any type of conductive gel to the skin of a user.

As briefly discussed above when BADS 100 is in use, the sense and reference electrodes within each differential sensor pair (e.g., 103, 105, 107, etc.) may be positioned on or in close proximity to different locations of the body (e.g., head) of a user. Thereafter, the sense and reference electrodes may produce electrical signals indicative of brain activity in regions of the user's brain that are proximate their respective locations. More specifically, the sense and reference electrodes in each differential sensor pair produce sense and reference signals, respectively, which are indicative of user brain activity proximate to their respective locations. It is noted that for the sake of the present disclosure, the terms “sense signal” and “reference signal” are used to refer to signals produced by different electrodes located on or proximate to different portions of the body of a user (e.g., different parts of the head). Thus, a “reference” signal is still a signal that is produced in response to detection of EEG signals by an electrode.

As will be described in greater detail later, in some embodiments the sense and reference electrodes within sensor block may be integral with or coupled to an apparatus that may facilitate their correct placement with regard to the head of a user. For example, in some embodiments the sense and reference electrodes may be integral with or coupled to a wearable device (e.g., eyewear, a helmet, headset, a hat, clothing, or the like) a sleep accessory (e.g., a mattress, a pillow, a sheet, a pillow case, a mattress pad, or the like), or some combination thereof. In any case, apparatus may be configured such that sense and reference electrodes of sensor block 101 are positioned appropriately with regard to a body part (head) of the user.

Returning to the example of FIG. 1, first sense electrode S₁ and first reference electrode R₁ may each be configured to produce a corresponding first sense signal and first reference signal (generally indicated by an arrow in FIG. 1) which are indicative of brain activity at their respective locations. Likewise, the sense and reference electrodes in the second and third differential sensor pairs may produce corresponding sense and reference signals. That is, second sense and reference electrodes S₂, R₂ may be configured to produce second sense and reference signals, respectively, which are indicative of brain activity at their respective locations. Moreover the third sense and reference electrodes S₃, R₃ may be configured to produce third sense and reference signals, respectively, which are indicative of brain activity at their respective locations. Without limitation, in some embodiments the electrodes in each of the differential sensor pairs 103, 105, 107 are non-contact electrodes, such as capacitive electrodes.

Without limitation, in some embodiments the sense and reference signals produced by the sense and reference electrodes, respectively are analog signals. That is, the first sense and reference electrodes (S₁, R₁) may each be configured to produce first analog sense and first analog reference signals, respectively, the second sense and reference electrode (S₂, R₂) may each be configured to produce a second analog sense and second analog reference signals, respectively, and so forth.

As further shown in FIG. 1, the (e.g., analog) sense and reference signals produced by each differential sensor pair in sensor block 101 are provided to signal processing block 111. In that regard, sensor block 111 (or, more particularly, the differential sensor pairs/electrodes therein) is communicatively coupled to signal processing block 111, e.g., as shown by the arrows joining differential sensor pairs 103, 105, 107 to signal processing block 111. Such coupling may be achieved in any manner, such as via conductive traces, a flexible conductor such as a conductive fabric, or other electrical coupling means. Without limitation, in some embodiments the electrodes of in sensor block 101 are coupled to signal processing block 111 via a soft and/or flexible conductor, such as a conductive (e.g., silver, gold, conductive polymer, etc.) fabric.

In instances where capacitive electrodes are used as one or more sense or reference electrodes, the signals measured and produced by such electrodes may be quite small (e.g., on the order of microvolts) and therefore it may be desirable to place signal processing block 111 in close proximity to such electrodes so as to avoid an undesirable degradation in signal quality. In that regard, while FIG. 1 (and various other FIGS. depict signal processing block 111 as being discrete from sensor block 101 (or more, particularly, the differential sensor pairs therein), in some embodiments signal processing block may be integral with sensor block 101, and/or with one or more differential sensor pairs therein. In the latter case, for example, a plurality of signal processing blocks 111 may be used, wherein each differential sensor pair is coupled to a respective one of the plurality of signal processing blocks. Put in other terms, each differential sensor pair may further include a signal processing block that is integral therewith or coupled in close proximity thereto.

In general, signal processing block 111 is configured to perform signal processing operations on the (e.g., analog) sense and reference signals provided by one or more differential signal pairs. As will be described in further detail below, the signal processing operations may include the generation of at least one digital differential signal based at least in part on the (e.g., analog) sense and reference signals provided by at least one differential sensor pair.

More specifically, signal processing block 111 may be configured to receive (analog) sense and reference signals from sense and reference electrodes in one or more differential sensor pairs, as noted above. Subsequently, signal processing block 111 may produce one or more differential signals based at least in part on a received sense signal and a received reference signal, e.g., from one or more differential signal pairs. Production of a differential signal may involve, for example, subtracting a reference signal from a reference electrode of a differential sensor pair from a sense signal from a sense electrode in the same differential sensor pair.

For example in the embodiment of FIG. 1 signal processing block 111 may receive first, second, and third sense and reference signals from first, second, and third sense (S₁, S₂, S₃) and reference (R₁, R₂, R₃) electrodes, respectively. In response to receipt of such signals, signal processing block may produce first, second, and third differential signals by subtracting each reference signal from its corresponding sense signal. That is, processing block 111 may produce: a first differential signal by subtracting the first reference signal produced by the first reference electrode R₁ from the first sense signal produced by the first sense electrode S₁; a second differential signal by subtracting the second reference signal produced by the second reference electrode R₂ from the second sense signal produced by the second sense electrode S₂; and a third differential signal by subtracting the third reference signal produced by the third reference electrode R₃ from the second sense signal produced by the third sense electrode S₃.

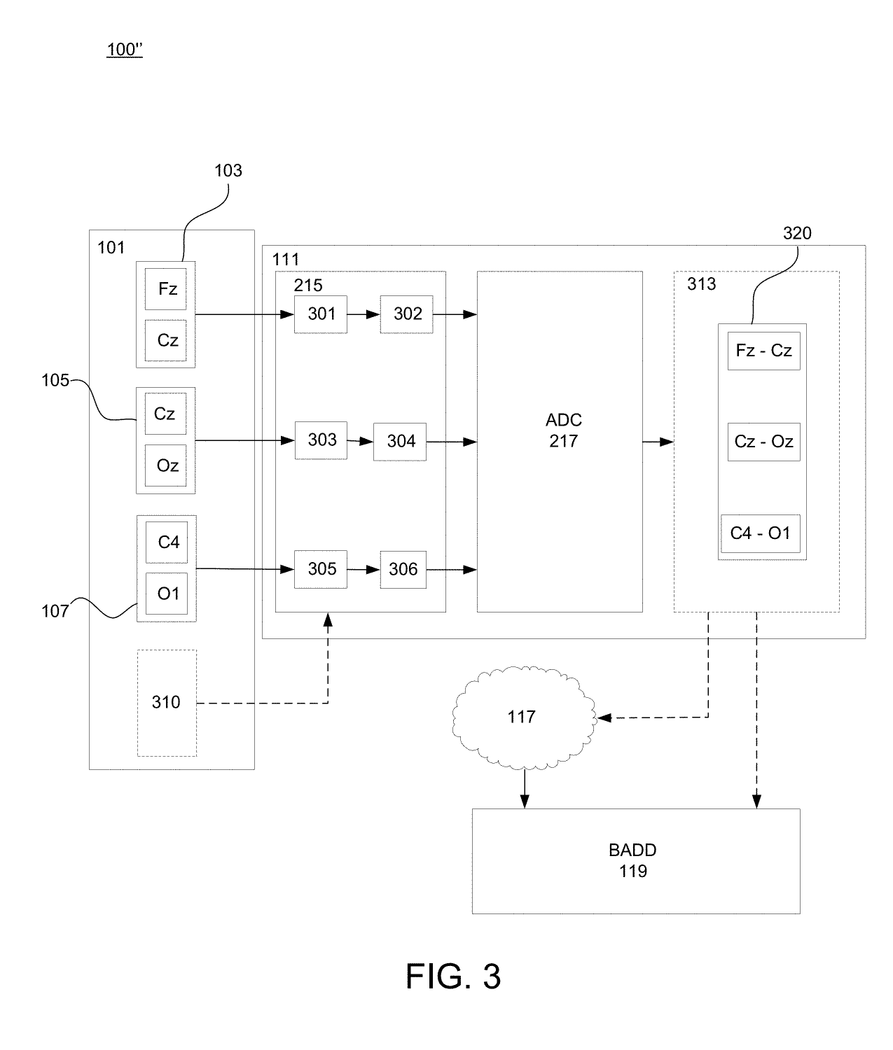

Alternatively in instances where the quality of one or more of the sense and/or reference signals is poor/inadequate (e.g., as determined by an optional microcontroller), signal processing block 111 may be configured to calculate a differential signal using one or more signals from one or more alternative electrodes, such as electrodes within or separate from another differential pair. This concept is shown in FIG. 3, which depicts another example of a BADS consistent with the present disclosure. As shown, BADS 101″ depicts sensor block 100 as including optional additional electrodes 310, which may provide an input signal that may be substituted for one or more of the sense and/or reference signals produced by one or more of the sense and/or reference electrodes.

In some embodiments signal processing block 111 may include or be in the form of circuitry that is configured to calculate one or more differential signals based at least in part on at least one received sense signal and at least one reference signal. In some embodiments signal processing block 111 includes circuitry that is configured to receive sense and reference signals from sense and reference electrodes in a differential sensor pair, and to produce a differential signal based at least in part on the received sense and reference signals. For example, signal processing block 111 may include one or more differential amplifier circuits that are configured to receive sense and reference signals (e.g., at different pins thereof) from sense and reference electrodes in a differential sensor pair, and to produce a differential signal based at least in part on the received sense and reference signals, as noted above. In instances where a plurality of differential sensor pairs are used (e.g., as shown in FIGS. 1-3), the signal processing block 111 may be configured to produce a differential signal from the reference and sense signals received from each of the plurality of differential sensor pairs.

As noted previously the sense and reference signals produced by the sense and reference electrodes in the differential sensor pairs of sensor block 101 may be quite small. This may be particularly true in instances wherein one or more of the sense and reference electrodes used is/are in the form of a capacitive electrode. Indeed as noted previously, capacitive electrodes may be used to accurately detect small changes in electric field produced by changes in the magnitude of action potentials within the brain. As the changes in the electric field detected by a capacitive electrodes is small (e.g., on the order of microvolts), the output (e.g., sense signal, reference signal) produced by such electrodes may be correspondingly small. It may therefore be desirable to amplify the sense and reference signals produced the sense and reference electrodes used in sensor block 100, so as to facilitate additional signal processing operations.

Therefore in some embodiments signal processing block 111 may include amplification circuitry that is configured to amplify received sense and/or reference signals, e.g., prior to or concurrent with the production of a differential signal as discussed above. For example, in instances where signal processing block includes one or more differential amplifiers, those differential amplifier(s) may be configured to amplify received reference and sense signals (e.g., received at different pins thereof), and to produce a differential signal based at least in part on the amplified reference and sense signals.

As noted previously sense and reference electrodes used in sensor block 100 are EEG electrodes that are utilized to EEG signals corresponding to brain activity of a user, and to produce corresponding sense and reference signals. In that regard it is noted that the frequency content of the sense and reference signals that corresponds to the detected EEG signals lies in a frequency range of about 1 to about 100 hertz (Hz), such as from about 1 to about 70 Hz. However, the sense and reference signals produced by the electrodes may include noise and other artifacts which may obscure relevant portions of such signals, potentially hindering the detection and/or processing of useful EEG information therein. For example, the sense and reference signals produced by the electrodes may include movement artifacts, power line noise (e.g. at 60 Hz, 50 Hz, etc.), combinations thereof, and the like, which may undesirably obscure desired components of a sense, reference, and/or differential signal.

Therefore in some embodiments signal processing block 111 includes filtration circuitry that is configured to reduce or eliminate undesirable noise from a sense, reference, and/or differential signal, such as line noise, movement artifacts, and the like. The filtration circuitry may also be configured to limit the frequency component of a signal to a desired frequency range, such as the frequency range of EEG signals that are of interest (in this case, from about 1 to about 100 Hz, such as about 1 to about 70 Hz). As the input sense and reference signals are generally small, such filtration circuitry in some embodiments is applied to filter an (amplified) differential signal, e.g., produced by the amplification circuitry discussed above. Of course, the filter circuitry need not be applied in that manner, and any suitable many of filtering may be employed.

Thus for example, in some embodiments signal processing block 111 includes circuitry to amplify received sense and reference signals (e.g., from one or more differential sensor pairs), and to produce (amplified) differential signal(s) therefrom. For example, in such embodiments signal processing block may include one or more differential amplifiers, as noted above. Although one differential amplifier (e.g. with many input pins) can be used for this purpose, in some embodiments signal processing block 111 includes a plurality of differential amplifiers, wherein each of the plurality of differential amplifiers is to operate on sense and reference signals provided by a respective one of the differential sensor pairs in sensor block 100. This concept is shown in the FIG. 3, which depicts signal processing block 111 as including amplifier and filtration circuitry 215 that includes a plurality of amplifiers 301, 303, 305 (e.g., differential amplifiers) that are each configured to produce a differential signal based at least in part on sense and reference signals provided by one of a plurality of differential sensor pairs.

In any case, the signal processing block 111 may further include filtration circuitry that functions to filter the (amplified) differential signal(s). For example, signal processing block may include one or more high and low pass filters so as to limit the frequency component of the differential signal(s) to within a defined range, such as from about 1 to 100 Hz, or even about 1 to about 70 Hz. Alternatively or additionally, the signal processing block may include one or more notch filters to remove line noise from the differential signal, e.g., at 50 Hz, 60 Hz, or the like. Of course, high pass, low pass, and notch filters are enumerated for the sake of example only, and any suitable filters may be used. In some embodiments, the filtration circuitry in signal processing block 111 may include a plurality of filter circuits, wherein each filter circuit is to operate on one of the differential signals produced as described above. This concept is shown in FIG. 3, which depicts signal processing block 111 as including amplifier and filtration circuitry 215 that includes a plurality of filters 302, 304, 306, wherein each of the filters 302, 304, 306 is to operate on a differential signal output from a corresponding one of a plurality of amplifiers 301, 303, 305 (e.g., differential amplifiers).

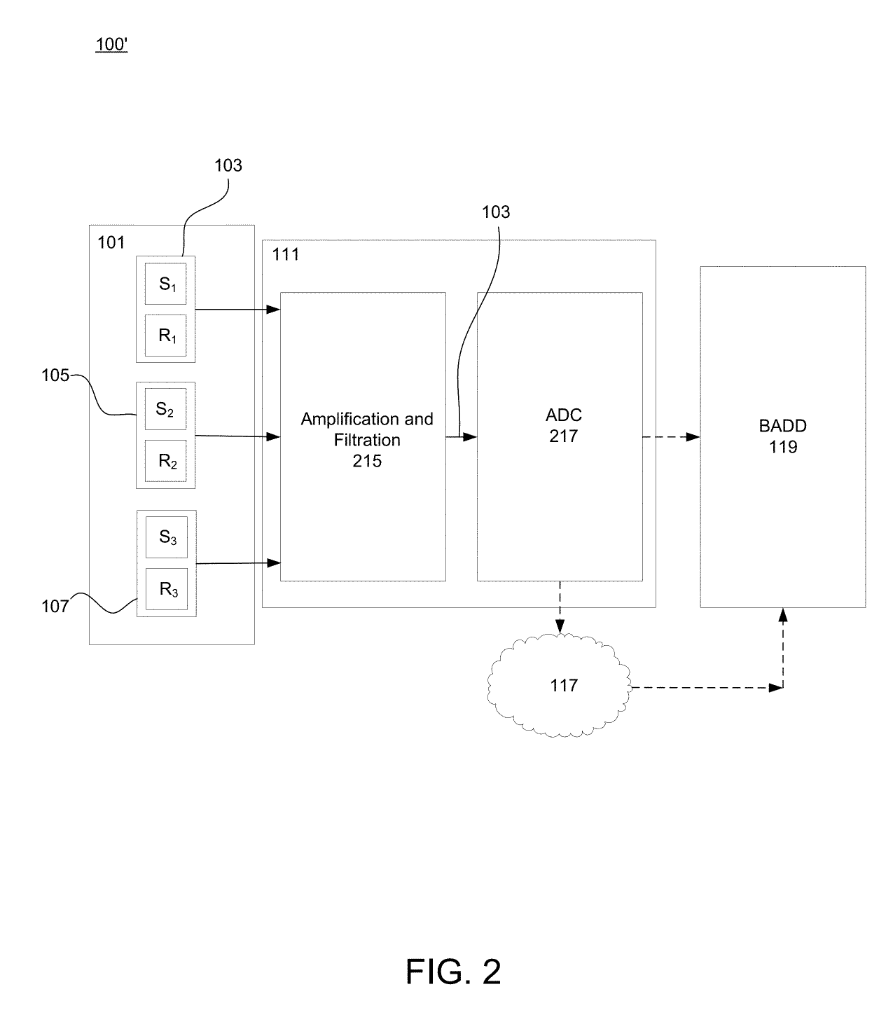

As noted previously the sense and reference signals produced by sense and reference electrodes in sensor block 101 may be analog signals. In such instances the signal processing block 111 may include amplification and filtration circuitry that is configured to amplify received analog sense and reference signals, produce one or more analog differential signals from the amplified analog sense and reference signals, and to filter the analog differential signal(s) to produce filtered analog different signals as generally discussed above. This concept is shown in FIGS. 2 and 3. As shown in those figs, BADS 100′, and BADS 100″ include all of the same components as BADS 100 of FIG. 1. However in the embodiments of FIGS. 2 and 3, processing block 111 includes amplification and filtration circuitry 215 and analog to digital converter (ADC) 217. Consistent with the foregoing discussion, the amplification and filtration circuitry 215 functions to amplify received analog sense and reference signals from one or more differential sensor pairs (S₁, R₁; S₂, R₂; S₃; R₃. etc.), produce one or more analog differential signals therefrom, and apply one or more filters to the analog differential signal(s).

To facilitate further processing the filtered analog differential signal(s) may be converted to one or more digital differential signals. In that regard, signal processing block 111 may include circuitry to convert one or more analog differential signals to a corresponding digital differential signals, wherein the digital differential signals are representative of brain activity of a user (e.g., proximate the locations of the sense and reference electrodes in the differential sensor pairs). For example, in some embodiments signal processing block 111 may include one or more analog to digital converters that function to convert analog differential signals to digital differential signals. This concept is shown in FIGS. 2 and 3, which depict a BADS 100′, 100″ with a signal processing block 111 that includes analog to digital converter (ADC) 217.

ADC 217 may function to convert analog signals received from amplifications and filtration circuitry, thereby producing a digital differential signal. It is noted that while FIGS. 2 and 3 depict embodiments in which a single ADC is used, it should be understood that such a configuration is not required. Indeed, the present disclosure envisions embodiments in which a plurality of ADC's are included in signal processing block 111, wherein each of the plurality of ADC's is to operate an respective one of a plurality of filtered analog differential signals, e.g., produced by filters 302, 304, 306, etc. at shown in FIG. 3.

In some instances, the at least one digital differential signal produced by the signal processing block 111 may be in a time (temporal) domain. In such instances, the at least one digital differential signal may be converted to a frequency domain, e.g., by signal processing block 111, an optional processing block 313 (as shown in FIG. 3) and/or brain activity detection device (BADD) 119, resulting in the production of at least one frequency domain signal that is representative of brain activity of a user. For example, in some embodiments signal processing block 111 may optionally include a microcontroller or other processor 313 (as shown in FIG. 3 which may function to perform a discrete Fourier Transform (DFT) or other single processing operations to convert one or more digital differential signals to a frequency domain. This concept is shown in FIG. 3, which depicts BADS 100″ as including a signal processing block 111 with an optional microcontroller 313. As shown, optional microcontroller 313 may receive digital differential signals 320 from ADC 217. Optional microcontroller 313 may perform DFT or other operations on the digital differential signals 320 to convert them to corresponding frequency domain signals. The frequency domain signals may then be conveyed to BADD 119, either directly or through network 117 (e.g., the Internet, wired or wireless communication, or the like), for further processing, as discussed later.

Of course the configuration shown in FIG. 3 is not limiting, and signal processing block 111 need not include microcontroller 313, or may include such a microcontroller for purposes other than converting digital differential signals to the frequency domain. In such instances, conversion of the digital differential signals may be performed using BADD 119 or other external processing capabilities. For example and as shown in FIG. 2, digital differential signals produced by ADC 217 may be conveyed to BADD 119, either directly or through network 117. In such instances, BADD 119 may configured to convert the received digital differential signals to the frequency domain. For example, in some embodiments BADD 119 may include memory storing computer readable instructions that, when executed by a processor of BADD 119, cause BADD 119 to convert a received digital differential signals to the frequency domain, e.g., using DFT or another signal processing technique. Alternatively or additionally, BADD 119 may include circuitry or logic implemented at least in part in hardware that is configured to convert received digital differential signals to the frequency domain.

Alternatively or additionally, conversion of digital differential signals to the frequency domain may be performed by other computing resources, such as but not limited to one or more servers (not shown). For example, processing block 111 may be configured to transmit digital differential signals via network 117 to one or more servers (not shown), for conversion to the frequency domain. The resulting frequency domain signals may then be transmitted to BADD 119 (e.g., directly or via network 117) for further analysis.

The resulting frequency domain signal(s) may be then be communicated to BADD 119 for processing. In that regard, BADD 119 may be configured to determine health related information about a user of the BADS based at least in part on one or more frequency domain signals. For example, BADD 119 may perform frequency analysis on one or more frequency domain signals to determine features thereof, and correlate such features to health related information, such as specific brain activity, user stress level, sleep state, etc. In instances where BADS 100 is used to perform sleep state analysis, for example, BADD 119 may be configured to perform frequency analysis on the frequency domain signal(s) to determine at least one dominant frequency thereof. BADD 119 may then determine whether the user is asleep and/or determine which sleep state a user is in, based at least in part on the dominant frequency(ies).

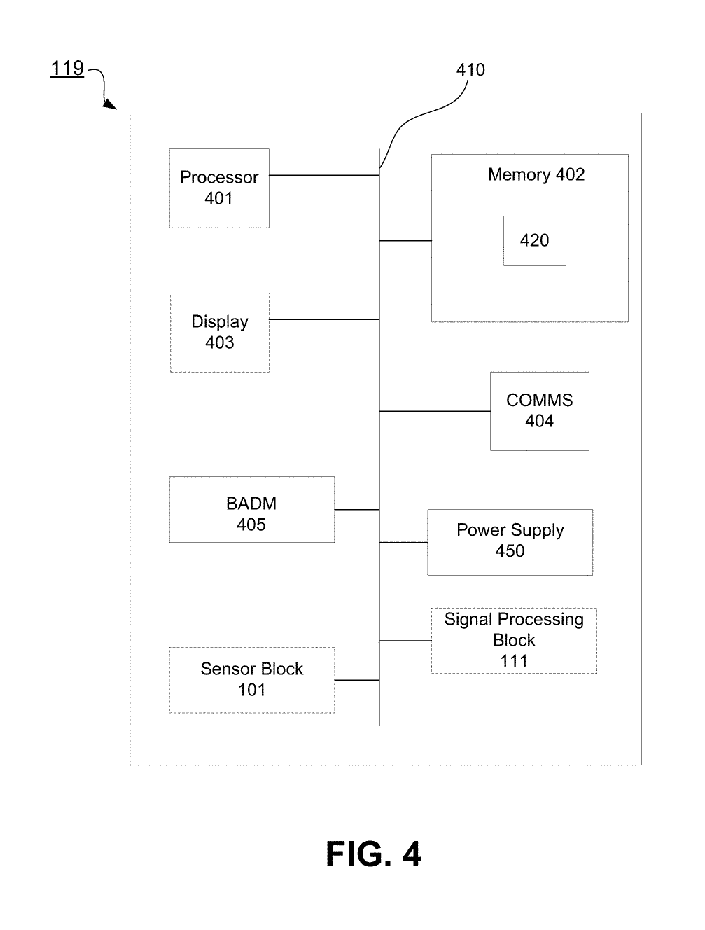

Reference is now made to FIG. 4, which is a block diagram of system architecture of the example of a brain activity detection device (BADD) consistent with the present disclosure. As shown, BADD 119 includes processor 401, memory 402, optional display 403, communications (COMMS) circuitry 404, and power supply 450, which may be in wired communication (e.g., via a bus or other suitable interconnects, not labeled) or wireless communication with one another.

It is noted that for the sake of clarity and ease of understanding, the various components of system BADD 119 are illustrated in FIG. 4 and are described herein as though they are part of a single electronic device, such as single mobile device or a single wearable device. It should be understood that this description and illustration are for the sake of example only, and that the various components of BADD 119 need not be incorporated into a single device. For example, the present disclosure envisions embodiments in which BADD 119 may be implemented in a device that is separate from processor 401, memory 402, optional display 403, and/or COMMS 404. Without limitation, in some embodiment BADD 119 is in the form of a mobile or other electronic device (e.g., a smart phone, a wearable device, a sleep accessory, etc.) that includes an appropriate device platform (not shown) that contains all of the components of FIG. 4.

Regardless of the form factor in which BADD 119 is implemented, processor 401 may be any suitable general purpose processor or application specific integrated circuit, and may be capable of executing one or multiple threads on one or multiple processor cores. Without limitation in some embodiments processor 401 is a general purpose processor, such as but not limited to the general purpose processors commercially available from INTEL® Corp., ADVANCED MICRO DEVICES®, ARM®, NVIDIA®, APPLE®, and SAMSUNG®. In other embodiments, processor 401 may be in the form of a very long instruction word (VLIW) and/or a single instruction multiple data (SIMD) processor (e.g., one or more image video processors, etc.). It should be understood that while FIG. 4 illustrates BADD 119 as including a single processor 401, multiple processors may be used.

Memory 402 may be any suitable type of computer readable memory. Example memory types that may be used as memory 402 include but are not limited to: semiconductor firmware memory, programmable memory, non-volatile memory, read only memory, electrically programmable memory, random access memory, flash memory (which may include, for example NAND or NOR type memory structures), magnetic disk memory, optical disk memory, combinations thereof, and the like. Additionally or alternatively, memory 402 may include other and/or later-developed types of computer-readable memory. Without limitation, in some embodiments memory 402 is configured to store data such as computer readable instructions in a non-volatile manner.

When used, optional display 403 may be any suitable device for displaying data, content, information, a user interface, etc., e.g. for consumption by a user of BADD 400 and/or BADS 100, 100′, 100″. Thus for example, optional display 403 may be in the form of a liquid crystal display, a light emitting diode (LED) display, an organic light emitting diode (OLED) display, a touch screen, combinations thereof, and the like.

COMMS 404 may include hardware (i.e., circuitry), software, or a combination of hardware and software that is configured to allow BADD 119 to receive and/or transmit data or other communications. For example, COMMs 404 may be configured to BADD 400 to receive signals from one or more differential sensor pairs (and/or electrodes therein), one or more components of processor block 111 (e.g., ADC 217, optional microcontroller 313, etc.), over a wired or wireless communications link (not shown), such as communications link complying with an 802.11, cellular communication, or other standard. Alternatively or additionally, COMMS 404 may enable system BADD 119 to send and receive data and other signals to and from another electronic device, such as another mobile or stationary computer system (e.g., a third party computer and/or server, a third party smart phone, a third party laptop computer, etc., combinations thereof, and the like, not shown). COMMS 404 may therefore include hardware to support wired and/or wireless communication, e.g., one or more transponders, antennas, BLUETOOTH™ chips, personal area network chips, near field communication chips, wired and/or wireless network interface circuitry, combinations thereof, and the like.

BADD 119 may be configured to monitor at least one biosignal of a user of BADS 100, 100′, 100″, and to determine health related information from the at least one biosignal. In that regard, in some embodiments BADD 119 includes brain activity detection module (BADM) 405. BADM 405 in some instances may be in the form of logic implemented at least in part in hardware to receive biosignals from BADS 100, 100′, 100″, which may be indicative of the brain and/or muscle activity of a user of device 100. Consistent with foregoing discussion, the at least one biosignal in some embodiments may be a digital differential signal produced by BADS 110, 100′, 100″. As previous described, the biosignals may be in the time or frequency domain. In the former case, BADM 405 may be configured to cause BADD 119 to convert the digital differential signal(s) to the frequency domain for analysis. For example, the BADM 405 may include computer readable instructions that when executed cause BADD 119 to convert a received digital differential signal in the temporal domain to the time domain.

In any case, BADM 405 may be further configured to cause BADD 119 to analyze biosignals (or, more specifically, frequency domain digital differential signals) in an effort to derive health related information therefrom. For example, BADM 405 may cause BADD 119 (or, more particularly, processor 401) perform frequency analysis operations on one or more frequency domain digital differential signals to determine a dominant frequency or frequency band thereof. Once the dominant frequency/frequency band of the digital differential signals has been determined, BADM 405 may cause BADD 119 to correlate one or more of determined dominant frequencies to one or more brain states of a user.

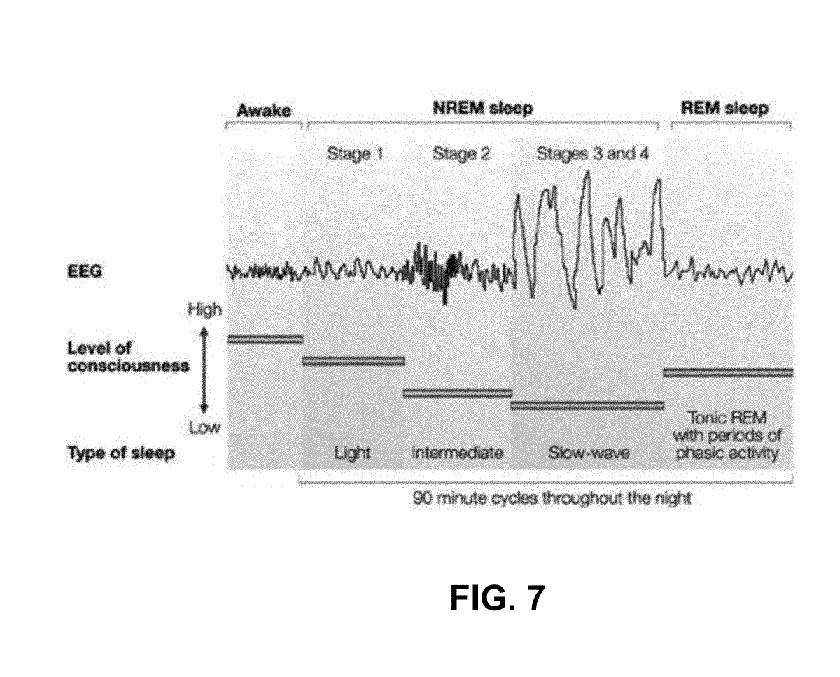

For example in the context of sleep monitoring, it has been shown that various stages of sleep are characterized by dominance of one or more frequency bands in an EEG signal. For example, and as shown in FIG. 7, different stages of sleep and/or awareness (e.g. alpha, beta, delta theta, edge of sleep, REM etc.) each have unique dominant frequency characteristics. For example the dominant frequency range of beta is 14-21 hz and indicates an actively awake state, the dominant frequency range of alpha is 8-13 Hz and indicates a relaxed awareness, the dominant frequency range of theta is 4-7 Hz and indicates drowsiness, edge of sleep, the dominant frequency of delta is <4 Hz and indicates dreaming and deep sleep INSERT. Accordingly, BADM 405 may include instructions that when executed cause BADD 119 (or, more specifically, processor 401) to determine dominant frequency bands from one or more frequency domain digital differential signals. Thereafter, BADM 405 may cause BADD 119 to determine which sleep state a user of BADS 100, 100′, 100″ is in (if any), and optionally for how long. This may be accomplished, for example, by comparing determined dominant frequencies/ranges to a lookup table 420 in memory 402, wherein the lookup table specifies a relationship between one or more dominant frequencies/frequency bands with one or more sleep stages (e.g., stage, 1, 2, 3, 4, Rapid eye movement, etc.). Once a sleep state of the user has been determined, BADM 405 may store a record of the determined sleep state in memory 402, and monitoring may continue if desired.

It is noted that while the foregoing discussion has focused on embodiments in which BADD 119 is provided as a separate component from one or more elements of BADS 100, 100′, 100′, such a configuration is not required. Indeed the present disclosure envisions embodiments in which one or more elements of BADS 100, 100′, 100″ are included in BADD 119. For example and as shown in FIG. 4, in some embodiments BADD 119 may optionally include sensor block 101, signal processing block 111, or a combination thereof. In such embodiments sensor block 101, signal block 111, and BADD 119 may function in substantially the same manner as described above, with the exception that transmission and/or communication pathways for the various signals may differ. For example, digital differential signals produced by signal processing block 111 may be transmitted to BADM 405 (and/or processor 401) for processing via a bus of BADD 119 (not labeled), as opposed to via wired or wireless communication as shown in FIGS. 1-3.

For the sake of clarity and ease of understanding, the present disclosure will now proceed to describe an example embodiment in which BADS 100″ is used to detect brain activity of a user. While this example focuses on the determination of which sleep state the user of BADS 100″ is in, it should be understood that the present disclosure is not limited to that application, as discussed above.

With the foregoing in mind reference is again made to FIG. 3, which is a block diagram of one example of a BADS 100″ consistent with the present disclosure. BADS 100″ includes sensor block 101, which as shown includes a plurality of differential sensor pairs 103, 105, 107. As note previously, each of the differential sensor pairs 103, 105, 107 include a sense electrode and a reference electrode. In use, the respective sense and reference electrodes in the differential sensor pairs 103, 105, 107 may be positioned proximate to various locations on a body of a user, such as a user's head. In operation, the first, second, and third differential sensor pairs 103, 105, 107 may be used to detect brain activity of a user of BADS 100″ over time, e.g., as the user is sleeping.

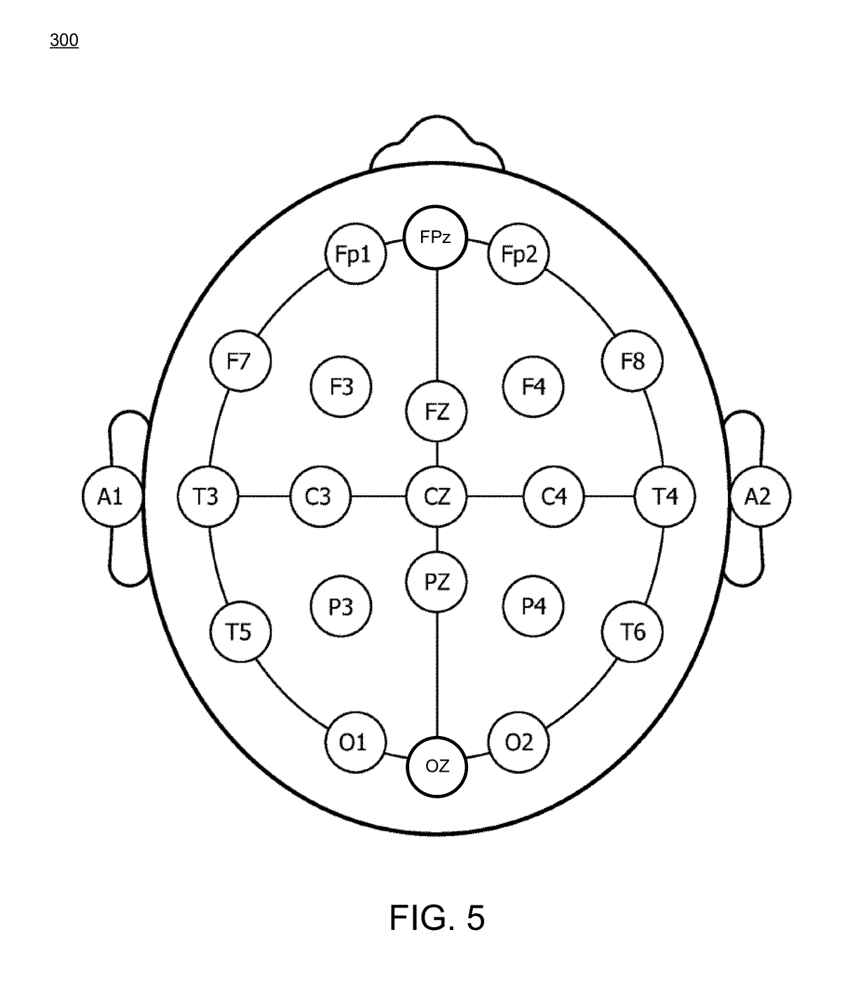

With regard to the positioning of the sense and reference electrodes reference is made to FIG. 5, which depicts an overview of electroencephalogram (EEG) electrode nomenclature under the “10-20 system”—an internationally recognized method to describe and apply the location of scalp electrodes in the context of EEG testing. The letters F, T, C, P and O stand for frontal, temporal, central, parietal, and occipital, respectively. Note that there is not central lobe, i.e., that the letter “C” is used for identification purposes only. Even numbers (2, 4, 6, 8) refer to electrode positions on the right hemisphere, whereas odd numbers (1, 3, 5, 7) refer to those on the left hemisphere. With that in mind, in the illustrated embodiment of FIG. 3 the first differential sensor pair 103 includes a sense electrode at the Fz location and a reference electrode at the Cz location. The second differential sensor pair 105 includes a (same or different) electrode at the Cz location, and a reference electrode at the Oz location. The third differential sensor pair 107 includes a sense electrode at the C4 location, and a reference electrode at the O1 location. Optional additional electrodes 310 may also be included at other locations. For example, optional electrodes 310 may include one or more electrodes at the Fpz, C3, O1, and M2 locations.

It is emphasized that the specific electrode locations specified in the embodiment of FIG. 3 are not critical or limiting, particularly in instances where BADS 100″ is used to perform sleep analysis. Indeed because sleep produces global changes in brain activity, the position of the electrodes in differential sensor pairs 103, 305, 107 is not important. Rather, the quality of the differential signals used may be important to determining sleep state. Accordingly, BADS 100″ may include an optional microcontroller 313 (as discussed above). One function of optional microcontroller 313 may be to select configure differential sensor pairs with pairs of sense and reference electrodes that provide a high quality differential signal. For example, If the Fz and Cz pairing in differential sensor pair 103 is considered to produce a signal that is relatively poor, microcontroller 313 may cause the substitution of an optional electrode 310 for the Fz or Cz electrode. Similar analyses may be performed my microcontroller 313 with regard to the quality of the differential signal produced by the Cz and OZ pairing in differential sensor pair 105, and the C4 and O1 pairing in differential sensor pair 107. In some embodiments for example, an electrode at the Fpz location may be substituted for the Fz electrode in differential sensor pair 103, an electrode at the C3 location may be substituted for the Cz or C4 electrodes in differential sensor pairs 105, 107, an electrode at the O2 location may be substituted for the O1 electrode etc.

In any case, in operation differential sensor pair 103 may produce first sense and reference signals, differential sensor pair 105 may produce second sense and reference signals, and third differential sensor pair 107 may produce third sense and reference signals, e.g., wherein the first, second, and third sense and reference signals are representative of brain activity detected at the locations corresponding to the sense and reference electrodes in each of the first, second, and third differential sensor pairs. The first, second, and third sense and reference signals are communicated to signal processing block 111 as generally shown. more particularly, the first sense and reference signals are communicated to a first amplifier 301, the second sense and reference signals are communicated to a second amplifier 303, and the third sense and reference signals are communicated to a third amplifier 305 within amplification and filtration circuitry 215 of signal processing block 111.

Consistent with the previous discussion, the first, second and third amplifiers 301, 303, 305 may be configured to amplify the first, second, and third sense and reference signals, respectively, and to produce corresponding first, second, and third (analog) differential signals. For example, the first, second and third amplifiers 301, 303, 305 may each be a differential amplifier that is configured to generate a differential signal by subtracting an input reference signal from an input sense signal. In this case, first amplifier 301 may produce a first differential signal by subtracting the first reference signal from the Cz electrode from the first sense signal from the Fz electrode. Likewise, the second and third amplifiers 303, 305 may produce second and third differential signals by subtracting a received input reference signal (e.g., from the Oz or O1 electrode) from a received input sense signal (e.g., from the Cz ore C4) electrode.

The first, second, and third differential signals may then be filtered, as discussed above. For example, one or more low pass, high pass, and/or notch filters may be applied to remove unwanted portions (e.g., noise) from first, second, and third differential signals. More particularly in the embodiment of FIG. 3, the first differential signal may be filtered by first filter 302, the second differential signal may be filtered by second filter 304, and the third differential signal may be filtered by third filter 306. In this non-limiting embodiment, the first, second, and third filters may include low and high pass filters that eliminate portions of the differential signal outside of about 1 to about 100 Hz (such as about 1 to about 100 Hz), and a notch filter to remove line noise, e.g., at 50 Hz or 60 Hz.

The resulting first, second, and third filtered analog differential signals may then be converted to corresponding first, second, and third digital differential signals by ADC 217, as discussed above. The resulting first, second, and third digital differential signals may then be converted from the temporal domain to the frequency domain as discussed above, e.g., by optional microcontroller 313 or BADD 119. BADD 119 may thereafter perform dominant frequency analysis on each of the first, second and third frequency domain digital differential signals, so as to determine the dominant frequency component(s) of each of those signals over time measurement period. BADD 119 may correlate determined dominant frequency components to sleep states of the user (e.g., stages 104, REM, etc.) as discussed above. For example, BADD 119 may compare determined dominant frequency components of the frequency domain digital signals over time to reference information in a lookup table 420, e.g., stored in a memory 402 thereof. The reference information may include reference dominant frequency components that are correlated with specific sleep states.

BADD 119 (or more particularly, BADM 405) may record determined sleep states over the course of the measurement, and may record those states and other data (e.g., time) in one or more data structures within memory 402. Alternatively or additionally, BADM 405 may cause the display of determined sleep states and other information, e.g., on optional display 403. Still further, determined sleep states and other information may be communicated to external computing resources (e.g. one or more servers, mobile devices, etc.), for reference and/or further analysis.

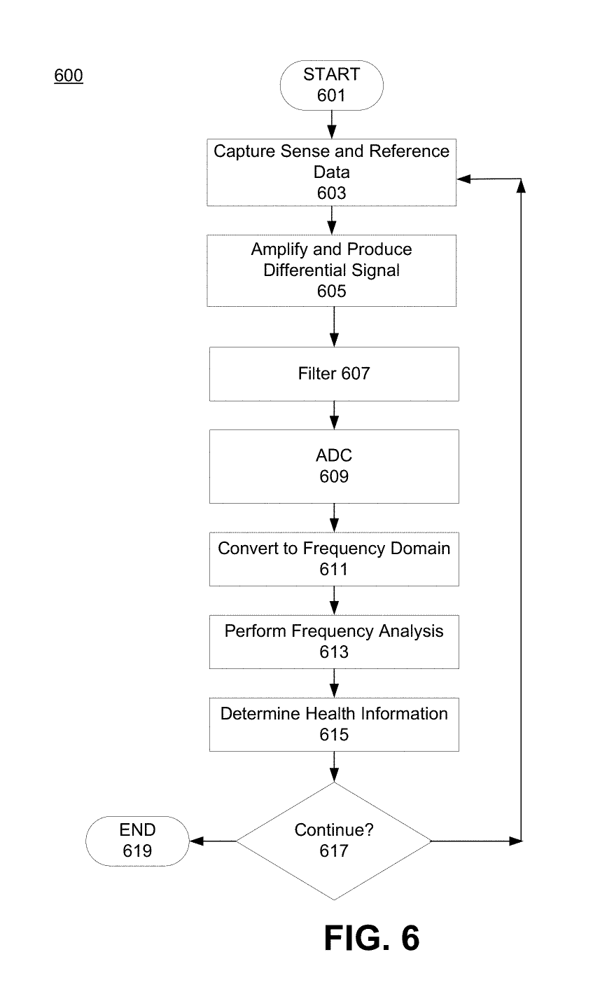

Another aspect of the present disclosure relates to methods of performing brain activity detection. In that regard reference is made to FIG. 6, which is a flow diagram of example operations of one example method of performing brain activity detection consistent with the present disclosure. As shown, the method 600 begins at block 601. The method may then proceed to block 603, pursuant to which sense and reference data may be captured from a user of a brain activity detection system. For example, sense and reference signals may be captured from one or a plurality of sense and reference electrodes in one or a plurality of differential sensor pairs, as described above.

The method may then proceed to block 605, pursuant to which signal processing operations are performed to amplify acquired sense and reference signals and to produce one or more (analog) differential signals, as described previously. The method may then proceed to block 607, pursuant to which the (analog) differential signals may be subject to signal processing operations to filter undesirable components therefrom. For example, operations pursuant to block 607 may include applying one or more high pass, low pass, and/or notch filters to remove noise from the (analog) differential signal(s) produced pursuant to block 605, and/or to limit the frequency component of the differential signal(s) to the a range containing relevant EEG information, e.g., from about 1 to about 100 Hz, or even from about 1 to about 70 Hz.

The method may then proceed to block 609, pursuant to which the filtered (analog) differential signal(s) may be converted to one or more digital differential signals, e.g., by an analog to digital converter (ADC), as discussed above. The resulting digital differential signal may be in the temporal domain. In such instances the method may proceed to block 611, pursuant to which the temporal domain digital differential signal(s) may be converted to the frequency domain, e.g., by the execution of a discrete Fourier Transform (DFT) operations by an optional microcontroller and/or a processor of a brain activity detection device, as discussed above.

The method may then proceed to block 613, pursuant to which frequency analysis may be performed on the frequency domain digital differential signal(s), and the dominant frequency component(s) thereof may be determined. The method may then proceed to block 615, pursuant to which health information (e.g., sleep state, length, brain activity, stress, etc.) may be determined, e.g., from the dominant frequency components determined from the frequency domain digital differential signal(s).

The method may then proceed to block 617, pursuant to which a determination may be made as to whether monitoring of a user is to continue. If so, the method may loop back to block 603. But if not, the method may proceed to block 619 and end.

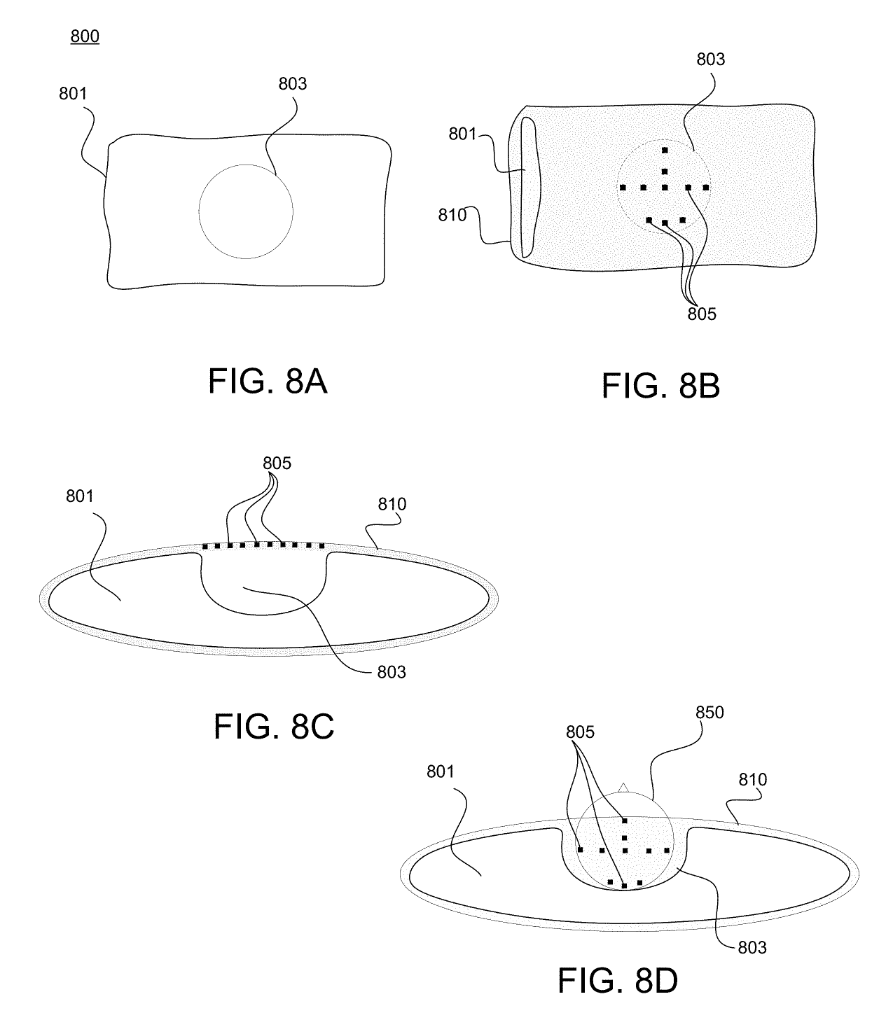

And aspect of the present disclosure relates to articles, devices, etc. that include one or more components of a brain activity detection system consistent with the present disclosure. In that regard, as noted above the brain activity detection systems may be implemented in the form of a wearable device (e.g., head ware), a sleep accessory (e.g., pillows, sheets, pillowcases, etc.), and the like. To demonstrate this concept reference is made to FIGS. 8A-8D, which depict various views of a sleep accessory including components of a brain activity detection system consistent with the present disclosure.

As shown, sleep accessory 800 includes a pillow 801 including a recess 803. A pillow cover 810 encompasses at least a portion of pillow 801. As best shown in FIG. 8C, pillow cover 810 extends over recess 803, and includes a plurality of electrodes 805. The electrodes 805 are positioned on or within pillow cover 810 such that they are disposed over or in proximity to recess 803, e.g., when pillow 801 is not in use by a user (e.g., a human).

Each of the plurality of electrodes 805 is or includes an EEG electrode, such as those described above. Without limitation, in some embodiments each of the plurality of electrodes 705 includes a capacitive electrode that can function to detect minute changes in the electrical activity of the brain, as noted above. The electrodes 805 may be electrically coupled to one another and/or to a signal processing block (e.g., in pillow 801 or in an external device, not shown), by an electrical conductor. For example, in some embodiments conductive fabric may be used to couple electrodes 805 to a signal processing block within pillow 801 or in an external device.

As best shown in FIG. 8D, recess 803 is generally configured to receive the head 850 of a user. As further shown, pillow cover 810 and recess 803 may be configured such that when a user lays his head within recess 803, pillow cover 810 stretches and electrodes 805 are positioned at various locations along the user's head. In instances where electrodes 805 are capacitive electrodes, they may then be used to detect EEG signals produced by the user, even if they are not in direct contact with the user's scalp. To facilitate detection of useful signals, the electrodes 805 may be arranged in a pattern or array on or within pillow cover 810. For example and as best shown in FIG. 8B, electrodes 805 may be arranged in a “plus” or other geometric configuration on or within pillow cover 810.

In operation, a microcontroller or another component (e.g., a processor of a brain activity detection device) may assign various of the electrodes 805 to one or more differential sensor pairs. Electrodes 805 assigned to those sensor pairs may then collect sense and reference signals from a user of sleep accessory 800, as discussed above in connection with FIGS. 1-3. The resulting differential signals may then be processed to produce digital differential signals that are representative of brain activity of the user, as previously discussed. For example, the differential signals may be processed into frequency domain digital differential signals, which may be subject to frequency analysis to determine which sleep state a user of sleep accessory 800 is in, and/or other factors relevant to the quality and quantity of the user's sleep.

### EXAMPLES

The following examples represent additional non-limiting embodiments of the present disclosure.

### Example 1

According to this example there is provided a brain activity detection system, including: a sensor block including at least one differential sensor pair, the at least one differential sensor pair including at least one reference electrode and at least one sense electrode, the reference and sense electrodes configured to produce analog reference and analog sense signals respectively, the analog reference and analog sense signals indicative of activity of a brain of a user; a signal processing block coupled to the sensor block, the signal processing block including circuitry to generate at least one digital differential signal based at least in part on the analog reference and analog sense signals, the at least one digital differential signal being in a time domain; and at least one processor in communication with the signal processing block, the at least one processor to convert the at least one digital differential signal in the time domain to a frequency domain digital differential signal, the frequency domain digital differential signal indicative of health information corresponding to the brain; wherein each of the at least one reference electrode and the at least one sense electrode includes a capacitive electrode.

### Example 2

This is example includes any or all of the features of example 1, wherein: the sensor block includes a plurality of differential sensor pairs, the plurality of differential sensor pairs to produce a corresponding plurality of sense and reference signals; and the signal processing block includes circuitry to generate a plurality of digital differential signals, wherein each of the plurality of digital differential signals corresponds to a respective one of the plurality differential sensor pairs.

### Example 3

This is example includes any or all of the features of any one of examples 1 and 2, wherein the signal processing block includes amplification circuitry to amplify the analog reference and analog sense signals and to produce an analog differential signal based at least in part on the amplified analog reference and analog sense signals.

### Example 4

This is example includes any or all of the features of example 3, wherein the amplification circuitry includes a differential amplifier, the differential amplifier to produce the analog differential signal at least in part by subtracting the amplified analog reference signal from the amplified analog sense signal.

### Example 5

This is example includes any or all of the features of any one of examples 3 and 4 wherein the signal processing block further includes filtration circuitry, the filtration circuitry to apply at least one filter to the analog differential signal.

### Example 6

This is example includes any or all of the features of example 5, further including an analog to digital converter to convert the analog differential signal to produce the digital differential signal in the temporal domain.

### Example 7

This is example includes any or all of the features of any one of examples 1 to 6, wherein the at least one processor is further to perform signal processing on the digital differential signal in the frequency domain to identify at least one dominant frequency component of the digital differential signal in the frequency domain.

### Example 8

This is example includes any or all of the features of example 7, wherein the at least one processor is further to determine the health information correlating to the brain based at least in part on the at least one dominant frequency component.

### Example 9

This is example includes any or all of the features of any one example 8, wherein the health information is a sleep state, stress, or other brain activity of the user.

### Example 10

This is example includes any or all of the features of example 2, wherein the plurality of sense and reference electrodes are present on or within a cover for a sleep accessory.

### Example 11

According to this example there is provided a method of detecting brain activity, including: producing, with a sense electrode and a reference electrode in a differential signal pair, at least one analog reference signal and at least one analog sense signal, the analog sense and reference signals indicative of activity of a brain of a user; generating, with circuitry of a signal processing block coupled to the at least one sense electrode, at least one digital differential signal based at least in part on the analog reference and analog sense signals, the at least one digital differential signal being in a time domain; and converting, with at least one processor in communication with the circuitry of the signal processing block, the at least one digital differential signal in the time domain to a frequency domain digital differential signal, the frequency domain digital differential signal indicative of health information corresponding to the brain; wherein each of the at least one reference electrode and the at least one sense electrode includes a capacitive electrode.

### Example 12

This is example includes any or all of the features of any one of example 11, wherein the sensor block includes a plurality of differential sensor pairs and the method further includes: producing, with sense and reference electrodes in the plurality of differential pairs, a plurality of sense and reference signals corresponding to each of the plurality of differential pairs; and generating, with the circuitry of the signal processing block a plurality of digital differential signals, wherein each of the plurality of digital differential signals corresponds to a respective one of the plurality differential sensor pairs.

### Example 13

This is example includes any or all of the features of any one of examples 11 and 12, wherein the circuitry of the signal processing block includes amplification circuitry, and the method further includes: amplifying, with the amplification circuitry, the analog reference and analog sense signals to produce amplified analog reference and analog sense signals; and producing, with the amplification circuitry an analog differential signal based at least in part on the amplified analog reference and analog sense signals.

### Example 14

This is example includes any or all of the features of example 13, wherein the amplification circuitry includes a differential amplifier, and producing the analog differential signal includes subtracting the amplified analog reference signal from the amplified analog sense signal.

### Example 15

This is example includes any or all of the features of any one of examples 13 and 14, wherein the signal processing block further includes filtration circuitry, and the method further includes: applying, with the filtration circuitry, at least one filter to the analog differential signal.

### Example 16

This is example includes any or all of the features of example 15, wherein the method further includes: converting, with an analog to digital converter, the analog differential signal to the digital differential signal in the temporal domain.

### Example 17

This is example includes any or all of the features of any one of examples 11 to 16, wherein the method further includes: performing signal processing on the digital differential signal in the frequency domain to identify at least one dominant frequency component of the digital differential signal in the frequency domain; and determining the health information correlating to the brain based at least in part on the at least one dominant frequency component.

### Example 18

According to this example there is provided at least one computer readable medium including instructions which when executed by a processor of a brain activity detection system cause the system to perform the following operations including: producing, with a sense electrode and a reference electrode in a differential signal pair, at least one analog reference signal and at least one analog sense signal, the analog sense and reference signals indicative of activity of a brain of a user; generating, with circuitry of a signal processing block coupled to the at least one sense electrode, at least one digital differential signal based at least in part on the analog reference and analog sense signals, the at least one digital differential signal being in a time domain; and converting, with at least one processor in communication with the circuitry of the signal processing block, the at least one digital differential signal in the time domain to a frequency domain digital differential signal, the frequency domain digital differential signal indicative of health information corresponding to the brain; wherein each of the at least one reference electrode and the at least one sense electrode includes a capacitive electrode.

### Example 19

This is example includes any or all of the features of example 18, wherein the sensor block includes a plurality of differential sensor pairs and the instructions when executed by the processor cause the system to perform the following operations including: producing, with sense and reference electrodes in the plurality of differential pairs, a plurality of sense and reference signals corresponding to each of the plurality of differential pairs; and generating, with the circuitry of the signal processing block a plurality of digital differential signals, wherein each of the plurality of digital differential signals corresponds to a respective one of the plurality differential sensor pairs.

### Example 20

This is example includes any or all of the features of any one of examples 18 and 19, wherein the circuitry of the signal processing block includes amplification circuitry, and the instructions when executed by the processor cause the system to perform the following operations including: amplifying, with the amplification circuitry, the analog reference and analog sense signals to produce amplified analog reference and analog sense signals; and producing, with the amplification circuitry an analog differential signal based at least in part on the amplified analog reference and analog sense signals.

### Example 21

This is example includes any or all of the features of example 20, wherein the amplification circuitry includes a differential amplifier, and producing the analog differential signal includes subtracting the amplified analog reference signal from the amplified analog sense signal.

### Example 22

This is example includes any or all of the features of any one of examples 20 and 21, wherein the signal processing block further includes filtration circuitry, and the instructions when executed by the processor cause the system to perform the following operations including: applying, with the filtration circuitry, at least one filter to the analog differential signal.

### Example 23

This is example includes any or all of the features of example 22, wherein the instructions when executed by the processor cause the system to perform the following operations including: converting, with an analog to digital converter, the analog differential signal to the digital differential signal in the temporal domain.

### Example 24

This is example includes any or all of the features of any one of examples 18 to 23, wherein the instructions when executed by the processor cause the system to perform the following operations including: performing signal processing on the digital differential signal in the frequency domain to identify at least one dominant frequency component of the digital differential signal in the frequency domain; and determining the health information correlating to the brain based at least in part on the at least one dominant frequency component.

### Example 25

This is example includes any or all of the features of example 24, wherein the health information includes a sleep state, stress, or other brain activity of the user.

### Example 26

According to this example there is provided at least one computer readable medium including instructions which when executed by a processor of a brain activity detection system cause the system to perform the method of any one of examples 11 to 17.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

What is claimed is:

1. A brain activity detection system, comprising:
a sensor block comprising at least one differential sensor pair, the at least one differential sensor pair comprising at least one reference electrode and at least one sense electrode, the reference and sense electrodes configured to produce analog reference and analog sense signals respectively, the analog reference and analog sense signals indicative of activity of a brain of a user;
a signal processing block coupled to the sensor block, the signal processing block comprising circuitry to generate at least one digital differential signal based at least in part on the analog reference and analog sense signals, the at least one digital differential signal being in a time domain; and
at least one processor in communication with the signal processing block, the at least one processor to convert the at least one digital differential signal in said time domain to a frequency domain digital differential signal, the frequency domain digital differential signal indicative of health information corresponding to said brain;
wherein each of said at least one reference electrode and said at least one sense electrode comprises a capacitive electrode.

2. The brain activity detection system of claim 1, wherein:
said sensor block comprises a plurality of differential sensor pairs, said plurality of differential sensor pairs to produce a corresponding plurality of sense and reference signals; and
said signal processing block comprises circuitry to generate a plurality of digital differential signals, wherein each of said plurality of digital differential signals corresponds to a respective one of the plurality differential sensor pairs.

3. The brain activity detection system of claim 1, wherein said signal processing block comprises amplification circuitry to amplify said analog reference and analog sense signals and to produce an analog differential signal based at least in part on the amplified analog reference and analog sense signals.

4. The brain activity detection system of claim 3, wherein said amplification circuitry comprises a differential amplifier, the differential amplifier to produce said analog differential signal at least in part by subtracting said amplified analog reference signal from said amplified analog sense signal.

5. The brain activity detection system of claim 3, wherein said signal processing block further comprises filtration circuitry, the filtration circuitry to apply at least one filter to said analog differential signal.

6. The brain activity detection system of claim 5, further comprising an analog to digital converter to convert said analog differential signal to produce said digital differential signal in said temporal domain.

7. The brain activity detection system of claim 1, wherein said at least one processor is further to perform signal processing on said digital differential signal in said frequency domain to identify at least one dominant frequency component of said digital differential signal in said frequency domain.

8. The brain activity detection system of claim 7, wherein said at least one processor is further to determine said health information correlating to said brain based at least in part on said at least one dominant frequency component.

9. The brain activity detection system of claim 8, wherein said health information is a sleep state of said user.

10. The brain activity detection system of claim 2, wherein said plurality of sense and reference electrodes are present on or within a cover for a sleep accessory.

11. A method of detecting brain activity, comprising:
producing, with a sense electrode and a reference electrode in a differential signal pair, at least one analog reference signal and at least one analog sense signal, the analog sense and reference signals indicative of activity of a brain of a user;
generating, with circuitry of a signal processing block coupled to the at least one sense electrode, at least one digital differential signal based at least in part on the analog reference and analog sense signals, the at least one digital differential signal being in a time domain; and
converting, with at least one processor in communication with the circuitry of the signal processing block, the at least one digital differential signal in said time domain to a frequency domain digital differential signal, the frequency domain digital differential signal indicative of health information corresponding to said brain;
wherein each of said at least one reference electrode and said at least one sense electrode comprises a capacitive electrode.

12. The method of claim 11, wherein said sensor block comprises a plurality of differential sensor pairs and the method further comprises:
producing, with sense and reference electrodes in said plurality of differential pairs, a plurality of sense and reference signals corresponding to each of said plurality of differential pairs; and
generating, with said circuitry of said signal processing block a plurality of digital differential signals, wherein each of said plurality of digital differential signals corresponds to a respective one of the plurality differential sensor pairs.

13. The method of claim 11, wherein said circuitry of said signal processing block comprises amplification circuitry, and the method further comprises:
amplifying, with said amplification circuitry, said analog reference and analog sense signals to produce amplified analog reference and analog sense signals; and
producing, with said amplification circuitry an analog differential signal based at least in part on the amplified analog reference and analog sense signals.

14. The method of claim 13, wherein said amplification circuitry comprises a differential amplifier, and producing said analog differential signal comprises subtracting said amplified analog reference signal from said amplified analog sense signal.

15. The brain activity detection system of claim 13, wherein said signal processing block further comprises filtration circuitry, and the method further comprises:
applying, with said filtration circuitry, at least one filter to said analog differential signal.

16. The method of claim 15, further comprising:
converting, with an analog to digital converter, said analog differential signal to said digital differential signal in said temporal domain.

17. The method of claim 11, further comprising:
performing signal processing on said digital differential signal in said frequency domain to identify at least one dominant frequency component of said digital differential signal in said frequency domain; and
determining said health information correlating to said brain based at least in part on said at least one dominant frequency component.

18. At least one computer readable medium comprising instructions which when executed by a processor of a brain activity detection system cause the system to perform the following operations comprising:
producing, with a sense electrode and a reference electrode in a differential signal pair, at least one analog reference signal and at least one analog sense signal, the analog sense and reference signals indicative of activity of a brain of a user;
generating, with circuitry of a signal processing block coupled to the at least one sense electrode, at least one digital differential signal based at least in part on the analog reference and analog sense signals, the at least one digital differential signal being in a time domain; and
converting, with at least one processor in communication with the circuitry of the signal processing block, the at least one digital differential signal in said time domain to a frequency domain digital differential signal, the frequency domain digital differential signal indicative of health information corresponding to said brain;
wherein each of said at least one reference electrode and said at least one sense electrode comprises a capacitive electrode.

19. The at least one computer readable medium of claim 18, wherein said sensor block comprises a plurality of differential sensor pairs and said instructions when executed by said processor cause said system to perform the following operations comprising:
producing, with sense and reference electrodes in said plurality of differential pairs, a plurality of sense and reference signals corresponding to each of said plurality of differential pairs; and
generating, with said circuitry of said signal processing block a plurality of digital differential signals, wherein each of said plurality of digital differential signals corresponds to a respective one of the plurality differential sensor pairs.

20. The at least one computer readable medium of claim 18, wherein said circuitry of said signal processing block comprises amplification circuitry, and said instructions when executed by said processor cause said system to perform the following operations comprising:
amplifying, with said amplification circuitry, said analog reference and analog sense signals to produce amplified analog reference and analog sense signals; and
producing, with said amplification circuitry an analog differential signal based at least in part on the amplified analog reference and analog sense signals.

21. The at least one computer readable medium of claim 20, wherein said amplification circuitry comprises a differential amplifier, and producing said analog differential signal comprises subtracting said amplified analog reference signal from said amplified analog sense signal.

22. The at least one computer readable medium of claim 20, wherein said signal processing block further comprises filtration circuitry, and said instructions when executed by said processor cause said system to perform the following operations comprising:
applying, with said filtration circuitry, at least one filter to said analog differential signal.

23. The at least one computer readable medium of claim 22, wherein said instructions when executed by said processor cause said system to perform the following operations comprising:
converting, with an analog to digital converter, said analog differential signal to said digital differential signal in said temporal domain.

24. The at least one computer readable medium of claim 18, wherein said instructions when executed by said processor cause said system to perform the following operations comprising:
performing signal processing on said digital differential signal in said frequency domain to identify at least one dominant frequency component of said digital differential signal in said frequency domain; and
determining said health information correlating to said brain based at least in part on said at least one dominant frequency component.

25. The at least one computer readable medium of claim 24, wherein said health information comprises a sleep state of said user.
